(19) United States
(12) Patent Application Publication
(10) Pub. No.: US 2008/0300404 A1
(43) Pub. Date: Dec. 4, 2008

(54) Process for the Preparation of Mycophenolate Mofetil

(75) Inventors: Abdurrazzaque Mirajkar, Winnipeg (CA); Rudolf Kubela, Oakville (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: APOTEX FERMENTATION INC., Winnipeg, MB (CA)

(21) Appl. No.: 11/795,647

(22) PCT Filed: Jan. 19, 2006
(86) PCT No.: PCT/CA06/00061

§ 371 Date: Jul. 19, 2007

(30) Foreign Application Priority Data
Jan. 20, 2005 (CA) 2,493,508
Jan. 19, 2006 (CA) 2,533,326

(51) Int. Cl. C07D 413/12

(52) U.S. Cl. 544/153

(57) ABSTRACT

A process of manufacturing mycophenolate mofetil comprising reacting an alkyl ester of mycophenolic acid, with 2-(4-morpholinyl)ethanol in the presence of a catalyst selected from a form of zinc selected from metallic zinc or at least one zinc salt or at least one zinc oxide, or a form of calcium selected from metallic calcium or at least one calcium salt or at least one calcium oxide.

### FIELD OF THE INVENTION

The present invention relates to an improved process for preparing Mycophenolate Mofetil.

### BACKGROUND OF THE INVENTION

Mycophenolate mofetil [6-(4-hydroxy-6-methoxy-7-methyl-3-oxo-5-isobenzofuranyl)-4-methyl-4-hexenoate 2-(4-morpholinyl)ethyl ester, 3] is an effective immunosuppressive drug marketed under the name CellCept®.

Common synthetic routes to mycophenolate mofetil typically involve the esterification of mycophenolic acid (1) with 2-(4-morpholinyl)ethanol (2) as depicted in Scheme 1.

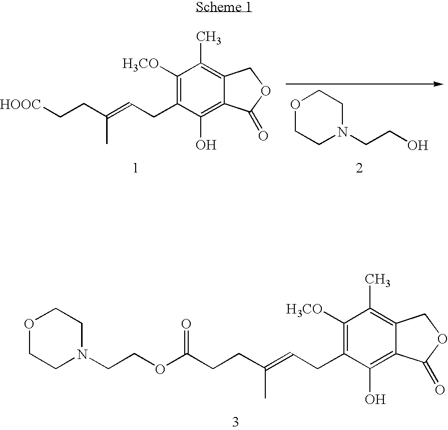

For instance, U.S. Pat. No. 4,753,935 discloses a process whereby 2-(4-morpholinyl)ethanol is condensed with mycophenolic acid using N,N′-dicyclohexylcarbodiimide (DCC) as an activating agent or by formation of the acid chloride form of mycophenolic acid. These two routes suffer from the fact they generate impurities such as a dimeric impurity. Also, when using DCC, the toxic N,N′-dicyclohexylurea by-product of the reaction is difficult to remove and DCC itself is highly allergenic. This patent also discloses a ‘less preferred’ third transesterification route (i.e., starting from an ester of mycophenolic acid) but does not elaborate any further.

The preparation of mycophenolate mofetil via the direct esterification of mycophenolic acid in organic solvents that are capable of removing water during the course of the reaction is also disclosed in various patents. For instance, U.S. Pat. No. 5,247,083 teaches the use of toluene, xylene, dichloromethane and WO 02100855 teaches the use of high-boiling dialkyl ethers such as dibutyl ether These routes suffer from the various disadvantages including long reaction periods and formation of coloured mycophenolate mofetil.

WO 03042393 teaches the use of an enzyme catalyst to achieve the esterification of mycophenolic acid with 2-(4-morpholinyl)ethanol. The use of enzymatic catalysis on an industrial scale poses difficulty, for instance in terms of volume, work-up and overall cost.

US 2004/0167130 disclosed a process for making mycophenolate mofetil comprising the transesterification of a lower carbon alkyl ester of mycophenolic acid with 2-(4-morpholinyl)ethanol using a catalyst consisting of an alkaline or alkaline earth metal salt, tin oxide or stannous oxide. This method suffers from the disadvantage that the invention only uses the preferred catalyst, dibutyl tin oxide, which is highly toxic, an irritant and expensive. Furthermore, the only example in US 2004/0167130 uses dibutyl tin oxide as a catalyst. No other alkaline or alkaline earth metal catalyst is used, and no other examples are found in the application.

WO 2004/089946 employs microwave irradiation to facilitate the reaction. However this requires specialized equipment on the industrial scale. Also, the reported yields in the six examples were low (24% to 57%).

WO 2005/023791 describes the making of mycophenolate mofetil, the patent appears to be a modification of U.S. Pat. No. 4,753,935 replacing thionyl chloride with oxalyl chloride. However, the method suffers due to poor yield and low purity of the final product.

The synthesis of mycophenolate mofetil by transesterification using catalysts, other than those described in US 2004/0167130, is unknown. While there is literature regarding the use of zinc and calcium in transesterification reactions of some specialized substrates such as β-keto esters (European Journal of Organic Chemistry, 2000, (8), pp. 1633-1635; Catalysis Letters, 62, (1999), pp. 67-69; Tetrahedron Letters (2002) 43, pp. 8583-8586) and phytosterol esters (Green Chemistry, 2003, 5(1), pp. 89-91), transesterification for the synthesis of mycophenolate mofetil using a catalyst selected from a form of zinc selected from metallic zinc or at least one zinc salt or at least one zinc oxide, or a form of calcium selected from metallic calcium or at least one calcium salt or at least one calcium oxide have not been described.

Thus, work was undertaken to overcome the deficiencies of the prior art to provide a facile and commercially viable process to produce mycophenolate mofetil on the industrial scale.

### SUMMARY OF THE INVENTION

An improved synthesis for the manufacture of mycophenolate mofetil via transesterification of a mycophenolate alkyl ester, preferably a lower alkyl ester selected from a C₁-C₄ alkyl ester, more preferably a methyl ester, with 2-(4-morpholinyl)ethanol in the presence of a catalyst selected from zinc (class IIB) or calcium (class IIA), its salts or oxides. We found that each of zinc and calcium, its salts or its oxides to be efficient promoters (catalysts) for this transesterification reaction and furnished mycophenolate mofetil in high yield and essentially free of impurities. If a salt is used, the preferred salt is the readily available zinc acetate for zinc, and the preferred salt is the readily available calcium chloride for calcium. Preferably, this reaction is carried out using from about 1 to 6 equivalents of 2-(4-morpholinyl)ethanol with or without a solvent at a temperature in the range of from about 70° C. to about 160° C. In one embodiment, the preferred range is from about 80° C. to about 120° C., in another embodiment the preferred range is from about 90° C. to about 130° C. and in yet another embodiment the preferred range is from about 100 to about 110° C. If a solvent or solvents is/are used, the solvent is preferably a nonprotic solvent such as toluene, xylene or higher boiling ethers, even more preferably the solvent is an excess of morpholinyl ethanol. From about 8 to about 48 hours, preferably from about 10 to about 48 hours and even more preferably from about 30 to about 40 hours such that the reaction is essentially complete (>97%). Product related impurities greater than 0.2-0.3% that are formed are eliminated during the work-up of the process. Some related impurities before the work-up were the unreacted methyl ester (0.5-1.8%) and some hydrolyzed ester to mycophenolic acid (1-3%) which were reduced to less than 0.10% during work-up.

However, pharmaceutical standards do not consider impurities at a level below <0.10% as significant.

According to one aspect of the invention, there is provided a process of manufacturing mycophenolate mofetil comprising reacting an alkyl ester of mycophenolic acid preferably a lower alkyl ester selected from a C₁-C₄ alkyl ester, and most preferably a methyl ester, with 2-(4-morpholinyl)ethanol in the presence of a catalyst selected from a form of zinc or calcium selected from metallic zinc or metallic calcium, or at least one zinc or calcium oxide or at least one zinc or calcium salt. Preferably the process comprises from about 1 to about 6 equivalents of 2-(4-morpholinyl)ethanol. Preferably the amount of the zinc or calcium or at least one zinc or calcium salt or at least one zinc or calcium oxide is from about 0.1 to about 3 equivalents, in another embodiment it is from about 0.5 to about 3 equivalents. Preferably the zinc salt is zinc acetate or zinc chloride. Preferably the zinc oxide is zinc oxide. Preferably the calcium salt is calcium chloride. Preferably the calcium oxide is calcium oxide. Preferably the process further comprises at least one solvent selected from the group consisting of a nonprotic solvent such as toluene, xylene or higher boiling ethers, even more preferably the solvent is an excess of morpholinyl ethanol. Preferably the process is conducted at a temperature in the range of from about 70° C. to about 160° C., more preferably in the range of from about 80° C. to about 120° C., even more preferably in the range of from about 100° C. to about 110° C., and even more preferably is the range of from about 90° C. to about 130° C.

Preferably the process further comprises the isolation of mycophenolate mofetil via standard techniques, preferably comprising solvent removal in vacuo, dilution in a solvent (preferably toluene or butyl acetate or ethyl acetate), filtering the catalyst off, washing the reaction solution, with water or with a base, preferably a sodium bicarbonate solution adding an aqueous acid, preferably HCl or sulphuric acid and extracting the product into the aqueous layer, basifying the aqueous phase and re-extracting or filtering the product into a solvent, concentrating the organic phase, crystallization and filtration of the final product.

In one embodiment, during the basification the pH is adjusted to the range of about 6 to about 7 to precipitate the product.

According to another aspect of the invention there is provided the use of a form of zinc or calcium selected from metallic zinc or calcium or at least one zinc or calcium salt or at least one zinc or calcium oxide in the manufacture of mycophenolate mofetil. If zinc or calcium or their salt or their oxide is not used, the reaction proceeds but at a much slower rate.

According to yet another aspect of the invention there is provided a process of manufacturing mycophenolate mofetil which does not require or requires minimal removal of colour from the final product.

Thus, the use of reagents of this type for the efficient synthesis of this valuable medicament represents a valuable invention and overcomes deficiencies of prior art methods.

### EXAMPLE 1

### Preparation of Mycophenolate Mofetil Using Zinc Acetate as Catalyst

Methyl mycophenolate (10 g) was suspended in of 2-morpholinoethanol (20 g) with of zinc acetate (10 g). The mixture was heated 90-100° C. and kept at this temperature for 18 hours under slightly reduced pressure (80-100 Torr below atm. pressure) to constantly remove the forming methanol. HPLC showed nearly complete conversion of methyl mycophenolate to mycophenolate mofetil (97.8% mofetil ester, 0.5% methyl ester and 1.2% of mycophenolic acid.

### EXAMPLE 2

### Preparation of Mycophenolate Mofetil from Mycophenolic Acid

Thionyl chloride (6.8 mL) was added over a period of 5 minutes to a solution of mycophenolic acid (25 g) in toluene (200 mL), after which 0.25 g of DMF was added and the mixture stirred for 2 hours at 20-25° C. The excess thionyl chloride with approximately 20% of toluene was removed under reduced pressure at 50° C. To the remaining stirred mixture was added methanol (50 mL) over a period of 5 minutes at 20-25° C. The mixture was stirred for an additional 30 minutes, after which TLC and HPLC indicated all the mycophenolic acid was converted to its methyl ester. The remaining solvents were removed under reduced pressure at 50° C. to afford a syrupy mass, to which 2-morpholinoethanol (51 g) and zinc acetate (25.6 g) were added. The mixture was heated to 90-100° C. under slightly reduced pressure for 32 hours. HPLC indicated 95.4% mofetil ester, 1.4% methyl ester and 2.99% mycophenolic acid with no additional related impurities.

### EXAMPLE 3

### Preparation of Mycophenolate Mofetil using Zinc Oxide as Catalyst

A mixture of methyl mycophenolate (10 g), 2-morpholino ethanol (20 g) and zinc oxide (4 g) was heated to 90-100° C. under slightly reduced pressure for 32 hours. The reaction was processed by addition of ethyl acetate followed by removal of the catalyst by filtration. The ethyl acetate layer was washed with water and then the organic phase was concentrated in vacuo to provide 11.5 g of the mycophenolate mofetil having a purity of 96.6% and 2.4% mycophenolic acid and 0.8% methyl mycophenolate.

### EXAMPLE 4

### Preparation of Mycophenolate Mofetil from Mycophenolic Acid Using Zinc Oxide

To a mixture of 100 g of mycophenolic acid and 500 mL of toluene, 43.0 g of thionyl chloride was added over a period of 10 minutes at ambient temperature, followed by addition of 0.1 g of dimethylformamide. After stirring the reaction mixture for two hours at 20-25° C., 200 mL of methanol was added over a period of 5 minutes. After 15 minutes of stirring a sample from the reaction mixture was checked by HPLC, which showed the presence of methyl mycophenolate only. All the volatiles were removed at reduced pressure at 50° C., to the resulting viscous residue 200 g of morpholino ethanol and 38 g of zinc oxide were added. The mixture was stirred at 100-110° C. under slightly reduced pressure for 35 hours after which it was cooled to 60° C., 500 mL of ethyl acetate was added, cooled to ambient temperature and the zinc oxide filtered. After standard work-up of extractions with sodium bicarbonate solution, treating the solution with diluted hydrochloric acid and back-extraction into ethyl acetate, the final product was crystallized yielding 123 g (91%) of mycophenolate mofetil.

### EXAMPLE 5

### Preparation of Mycophenolate Mofetil Using Zinc Metal

A mixture of 22.0 g of methyl mycophenolate, 43.0 g of morpholino ethanol and 6.5 g of granular (20 mesh) zinc metal was heated to 100-110° C. under reduced pressure for 44 hours. After filtering the metallic zinc and standard work-up, 26.9 g (94.4%) of crystalline mycophenolate mofetil with a purity of 99.32% (with 0.27% mycophenolate acid, 0.20% of methyl mycophenolate) was obtained. HPLC results are found below.

### EXAMPLE 6

### Preparation of Mycophenolate Mofetil Using Zinc Metal

To a dry and clean flask was charged 588.56 g (4.4 moles) of 2-morpholino ethanol 500 g (1.49 moles) of mycophenolic methyl ester and 19.55 g (0.29 moles) of zinc metal powder (100 mesh). The content of the flask was heated and the temperature of the reaction was maintained at 125-130° C. for 35-40 hours under nitrogen. At the end of the reaction, the content of the flask was cooled to 60-70° C. The reaction mass was diluted with 4.5-5.0 L of ethyl acetate, and then filtered to remove the catalyst (zinc).

The ethyl acetate solution was washed with about 1000 ml of water. The aqueous layer was separated. To the ethyl acetate solution containing mycophenolate mofetil was added 4.5-5 L of water, and the pH of the solution was adjusted to 3-4 with 35% sulphuric acid. The aqueous layer containing mycophenolate mofetil sulphate salt was separated from the organic layer.

The pH of the aqueous solution was adjusted to pH 6-7 and mycophenolate mofetil was precipitated out. The resulting solid was filtered and washed with water.

The solid obtained was dissolved in ethyl acetate(2.5 L) at 60-70° C. and filtered to separate insolubles, concentrated and crystallized. It was then filtered and dried to give 525 g of pure product of pharmaceutical grade.

HPLC purity: 99.9%.

### EXAMPLE 7

### Preparation of Mycophenolate Mofetil Using Zinc Oxide

To a dry and clean flask was charged 294.28 g (2.24 moles) of 2-morpholino ethanol 250 g (0.74 moles) of mycophenolic methyl ester and 12.16 g (0.148 moles) of zinc oxide.

The content of the flask was heated and the temperature of the reaction was maintained at 125-130° C. for 40 hours under nitrogen. At the end of the reaction, the content of the flask was cooled to 60-70° C. The reaction mass was diluted with 2.25-2.5 L of ethyl acetate, and was filtered to remove the catalyst (zinc oxide).

The ethyl acetate solution was washed with about 500 ml of water. The aqueous layer was separated. To the ethyl acetate solution containing mycophenolate mofetil was added 2.25-2.5 L of water, and the pH of the solution was adjusted to 3-4 with 35% sulphuric acid. The aqueous layer containing mycophenolate mofetil sulphate salt was separated from the organic layer.

The pH of the aqueous solution was adjusted to pH 6-7 and mycophenolate mofetil was precipitated out. The resulting solid was filtered and washed with water.

The solid obtained was dissolved in ethyl acetate (1.5 L) at 60-70° C. and filtered to separate insolubles, concentrated and crystallized. It was then filtered and dried to give 261 g of pure product of pharmaceutical grade.

HPLC purity: 99.81%.

### EXAMPLE 8

### Preparation of Mycophenolate Mofetil Using Zinc Chloride

To a dry and clean flask was charged 29.45 g (0.224 moles) of 2-morpholino ethanol 25 g (0.074 moles) of mycophenolic methyl ester and 2 g (0.0146 moles) of anhydrous zinc chloride. The content of the flask was heated and the temperature was maintained at 115-120° C. for 38-40 hours. At the end of the reaction, the content of the flask was cooled and ethyl acetate was added. The ethyl acetate solution was filtered to separate the catalyst, washed with 50 ml of water and the aqueous layer was separated from organic layer. To the organic layer was added water (125-150 ml) and the pH of the solution was acidified to 3-4 with sulphuric acid (35%). The aqueous layer containing Mycophenolate mofetil sulphate salt was separated from organic layer.

The pH of the aqueous solution was adjusted to 6-7 and mycophenolate mofetil was precipitated. The precipitate was filtered and washed with 50 ml of water. The resulting solid was recrystallized from ethyl acetate (150 ml) to give 28 g of mycophenolate mofetil.

HPLC purity: 99.65%

### EXAMPLE 9

### Preparation of Mycophenolate Mofetil Using Calcium Metal

To a dry and clean flask was charged 29.45 g (0.224 moles) of 2-morpholino ethanol 25 g (0.074 moles) of mycophenolic methyl ester and 2.96 g (0.074 moles) of calcium metal. The mixture was heated under nitrogen to 125-130° C. At the end of reaction, the mixture was cooled and diluted with ethyl acetate (200-250 ml). The catalyst was separated by filtration. The ethyl acetate was washed with water.

Water was added (250 ml) and the pH was adjusted to 3-4. The aqueous layer containing mofetil was separated. To the aqueous layer, the pH was adjusted to 6-7 and mofetil was precipitated. The precipitate was filtered and washed with water. The resulting solid was crystallized from ethyl acetate to give 24 g of mycophenolate mofetil with HPLC purity 99.54%

### EXAMPLE 10

### Preparation of Mycophenolate Mofetil Using Calcium Oxide

To a dry and clean flask was charged 29.45 g (0.224 moles) of 2-morpholino ethanol 25 g (0.074 moles) of mycophenolic methyl ester and 1.2 g (0.0148 moles) of calcium oxide. The mixture was heated under nitrogen to 125-130° C. At the end of reaction, it was cooled and diluted with ethyl acetate (200-250 ml). The catalyst was separated by filtration. The ethyl acetate was washed with water.

Water was added (250 ml) and the pH was adjusted to 3-4. The aqueous layer containing mofetil was separated. To the aqueous layer, the pH was adjusted to 6-7 and mofetil was precipitated. The precipitate was filtered and washed with water. The resulting solid was crystallized from ethyl acetate to give 24.16 g of mycophenolate mofetil with HPLC purity 99.65%.

### EXAMPLE 11

### Preparation of Mycophenolate Mofetil Using Calcium Chloride

To a dry and clean flask was charged 29.45 g (0.224 moles) of 2-morpholino ethanol 25 g (0.074 moles) of mycophenolic methyl ester and 1.65 g (0.0148 moles) of calcium chloride. The mixture was heated under nitrogen to 125-130° C. At the end of reaction, it was cooled and diluted with ethyl acetate (200-250 ml). The catalyst was separated by filtration. The ethyl acetate was washed with water.

Water was added (250 ml) and the pH was adjusted to 3-4. The aqueous layer containing mofetil was separated. To the aqueous layer the pH was adjusted to 6-7 and mofetil was precipitated. The precipitate was filtered and washed with water. The resulting solid was crystallized from ethyl acetate to give 22.3 g of mycophenolate mofetil with HPLC purity 97.3%.

### EXAMPLE 12

### Preparation of Mycophenolic Acid Methyl Ester (MPME)

100 g of mycophenolic acid (MPA) was suspended in 1000 ml of methanol, containing 2.5 g of concentrated sulphuric acid. The mixture was warmed at 30-35° C. for eight hours. At the end of the reaction when (MPA/MPME <2%), it was cooled to 10° C. and filtered, washed with 25 ml of methanol to give 95 g of methyl ester.

### HPLC 98.75% MPA 1.22%

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

1. A process of manufacturing mycophenolate mofetil comprising reacting an alkyl ester of mycophenolic acid with 2-(4-morpholinyl)ethanol in a solvent in the presence of a catalyst selected from a form of zinc or calcium selected from metallic zinc, metallic calcium, at least one zinc salt, at least one calcium salt, at least one zinc oxide, and at least one calcium oxide.

2. The process of claim 1 wherein the 2-(4-morpholinyl)ethanol is present in an amount from about 1 equivalent to about 6 equivalents.

3. The process of claim 1 wherein the reaction is conducted in excess of 2-(4-morpholinyl)ethanol.

4. The process of any one of claims 1, 2 or 3 wherein the catalyst is present in an amount of from about 0.1 to about 3 equivalents.

5. The process of claim 4 wherein the catalyst is selected from a form of zinc or calcium selected from the group consisting of metallic zinc, metallic calcium, zinc oxide, calcium oxide, calcium chloride, zinc chloride, calcium acetate and zinc acetate.

6. The process of any one of claims 1 or 2 further comprising at least one solvent selected from the group consisting of a nonprotic solvent.

7. The process of claim 6 wherein said nonprotic solvent is selected from toluene, xylene and higher boiling ethers.

8. The process of any one of claims 1, 2, or 3 wherein the process is conducted at a temperature in the range of from about 70° C. to about 160° C.

9. The process of claim 8 wherein the process is conducted at a temperature in the range of from about 80° C. to about 120° C.

10. The process of claim 9 wherein the process is conducted at a temperature in the range of from about 90° C. to about 130° C.

11. The process of claim 10 wherein the process is conducted at a temperature of from about 100° C. to about 110° C.

12. The process of any one of claims 1, 2, or 3 wherein the alkyl ester of mycophenolic acid is a C₁ to C₄ alkyl.

13. The process of claim 12 wherein the C₁ to C₄ alkyl is methyl.

14. The process of any one of claims 1, 2, or 3 further comprising the isolation of mycophenolate mofetil.

15. The process of claim 14 wherein said isolation comprises standard isolation techniques.

16. The process of claim 15 wherein the isolation is via precipitation.

17. The process of claim 16 wherein the precipitation of mycophenolate mofetil is conducted at a pH in the range of about 6 to about 7.

18. The use of a form of zinc or calcium selected from metallic zinc, metallic calcium, at least one zinc salt, at least one calcium salt or at least one zinc oxide and at least one calcium oxide in the manufacture of mycophenolate mofetil.

19. A process of manufacturing mycophenolate mofetil comprising
i) converting mycophenolic acid to an alkyl ester, and
ii) reacting said alkyl ester with 2-(4-morpholinyl)ethanol in the presence of a form of zinc, calcium, metallic zinc, metallic calcium, at least one zinc salt, at least one calcium salt, at least one zinc oxide, and at least one calcium oxide to form mycophenolate mofetil.

20. The process of any one of claims 1, 2 or 19 wherein the catalyst is selected from metallic zinc, at least one zinc salt, and at least one zinc oxide.

21. The process of any one of claims 1, 2 or 19 wherein the catalyst is selected from metallic calcium, at least one calcium salt, and at least one calcium oxide.
